(19) United States
(12) Patent Application Publication
(10) Pub. No.: US 2009/0132010 A1
(43) Pub. Date: May 21, 2009

(54) SYSTEM AND METHOD FOR GENERATING COMPLEX BIOELECTRIC STIMULATION SIGNALS WHILE CONSERVING POWER

(76) Inventor: James W. Kronberg, Aiken, SC (US)

Correspondence Address: SEED INTELLECTUAL PROPERTY LAW GROUP PLLC, 701 FIFTH AVE, SUITE 5400, SEATTLE, WA, 98104, US

(21) Appl. No.: 11/942,574

(22) Filed: Nov. 19, 2007

(51) Int. Cl. A61N 1/02

(52) U.S. Cl. 607/72

(57) ABSTRACT

A system and method for generating an electrical signal for use in biomedical applications may have power efficient features, support battery powered operation and, support a reduced risk of shock hazard. The system may include a controller for generating one or more control signals operable to control pulse generating and waveform processing circuits. The control signals may include at least two states alternating in a chosen pattern as a function of time. During at least one of the control signal states, an oscillator for generating a pulsed signal may be operable. During at least another of the control signal states, the oscillator can be disabled and completely shut off in order to conserve considerable power. The generated pulses may be processed to provide desired intensity and frequency components. The processed signals may be applied to biological material.

### TECHNICAL FIELD

The present disclosure relates to a pulsed signal generator for biomedical applications. In particular, the disclosure relates to a light-weight, compact pulsed signal generator that produces a complex bioelectric stimulation signal output waveform.

### BACKGROUND

Injuries, infections and degenerative conditions are major sources of pain, inconvenience, expense, lost work (and leisure) time, and diminished productivity. The problems associated with these conditions grow worse with age, since an injury which would heal quickly in a young, healthy person takes much longer in one who is older, in poor health, or both. In demographically-aging societies such as now seen in most of the industrialized nations, these social and economic impacts will become increasingly magnified over the course of the next several decades.

While it is difficult to estimate the total cost of such conditions—leaving aside their impact on quality of life—the total surely amounts to many billions of dollars per year in the United States alone. For example, between five and ten million United States residents suffer from broken bones every year, with many of these cases involving multiple fractures. In a young, healthy patient, many fractures need to be immobilized in a cast for six weeks or more. Even after the cast is removed, the patient's activities are frequently restricted until the healed bone regains its full strength. In the elderly, in persons with poor health or malnutrition, in patients with multiple fractures, or in patients with conditions that impact healing processes, fractures usually heal more slowly. In some cases, the fractures do not heal at all, resulting in the conditions known as “nonunion” or “nonunion fracture” which sometimes persists for a lifetime.

As a result, an estimated quarter-million person-years of productivity are lost in the United States due to bone fractures alone. Similar statistics can be generated not only for other classes of traumatic injury, but also for chronic conditions such as osteoarthritis, osteoporosis, diabetic and decubitus ulcers, damaged ligaments, tendonitis, and repetitive stress injuries (including the conditions commonly known as “tennis elbow” and carpal tunnel syndrome).

Since the 1960s, it has been increasingly recognized that the human body generates a host of low-level electric signals as a result of injury, stress and other factors; that these signals play a necessary part in healing and disease-recovery processes; and that such processes can be accelerated by providing artificially-generated signals which mimic the body's own in frequency, waveform and strength. Such “mimic” signals have been shown to have many effects in the body, including helping to direct mobile cells such as fibroblasts and macrophages to sites where they are needed (galvanotaxis) and causing the release of cell growth factors such as transforming growth factor beta (TGF-b) and insulin-like growth factor (IGF). The results can include more rapid healing of skin and muscle wounds, including chronic ulcers such as those resulting from diabetes; the mending of broken bones, including most nonunion fractures; the regrowth of injured or severed nerves; the repair of tissues damaged by repetitive motion, as in tendonitis and osteoarthritis; and the reduction of swelling, inflammation, and pain, including chronic pain for which the usual drug-based treatments do not bring satisfactory relief.

Some of the body's signals, such as the “injury potential” or “current of injury” measured in wounds, are D.C. (direct current) only, changing slowly with time. It has been found that bone fracture repair and nerve regrowth are typically faster than usual in the vicinity of a negative electrode but slower near a positive one, where in some cases tissue atrophy or necrosis may occur. For this reason, most recent research has focused on higher-frequency, more complex signals often with no net D.C. component.

While most complex-signal studies to date have been performed on bone fracture healing, the commonality of basic physiological processes in all tissues suggests that the appropriate signals will be effective in accelerating many other healing and disease-recovery processes. Indeed, specific frequency and waveform combinations have been observed to combat osteoarthritis and insomnia. Such signals can also stimulate hair growth, reduce swelling and inflammation, fight localized infection, and increase speed of the healing of injured soft tissues including skin, nerves, ligaments and tendons. The signals can also relieve physical pain without the substituted discomfort of TENS (transcutaneous electric nerve stimulation), and also relieve psychological pain such as depression when applied transcranially. The relief of psychological pain apparently results from pacemaker-like action causing increased coherence in the brain waves.

FIG. 1A illustrates a schematic view of a waveform 20 which has been found effective in stimulating bone fracture healing, where a line 22 in FIG. 1A represents the waveform on a short time scale, a line 24 in FIG. 1B represents the same waveform on a longer time scale, levels 26 and 28 represent two different characteristic values of voltage or current, and intervals 30, 32, 34 and 36 represent the timing between specific transitions. Levels 26 and 28 are usually selected so that, when averaged over a full cycle of the waveform, there is no net D.C. component. In real-world applications, waveform 20 is sometimes modified in that all voltages or currents decay exponentially toward some intermediate level between levels 26 and 28, with a decay time constant usually on the order of interval 34. The result is represented by a waveform 38 in FIG. 1C.

In a typical commercially-available device for treating fracture nonunions, in which the desired signals are induced in tissue through pulsed electromagnetic fields (PEMF), interval 30 is about 200 microseconds, interval 32 about 30 microseconds, interval 34 about 5 milliseconds, and interval 36 about 60 milliseconds. Alternate repetition of intervals 30 and 32 generates pulse bursts 40, each of the length of interval 34, separated by intervals of length 36 in which the signal remains approximately at level 28. Each waveform 38 thus comprises rectangular waves alternating between levels 26 and 28 at a frequency of about 4400 Hz and a duty cycle of about 85%. The pulse bursts are repeated at a frequency of about 15 Hz alternating with periods of substantially no signal, resulting in a duty cycle of about 7.5%.

FIG. 2A illustrates a schematic view of a waveform 50 which has been found effective in relieving psychological conditions such as anxiety, depression and insomnia when applied transcranially, where a line 52 in FIG. 2A represents the waveform on a short time scale, a line 54 in FIG. 2B represents the same waveform on a longer time scale, a line 56 in FIG. 2C represents the same waveform on a still longer time scale, levels 62, 62a and 62b represent two different characteristic values of voltage or current, and intervals 64, 66, 68, 70, 72a, 72b, 74a and 74b represent the timing between specific transitions. Level 60 is normally made zero, and levels 62a and 62b are usually equal but opposite in polarity.

In a typical commercially-available device for treating depression and related conditions, in which pulsed electric field (PEF) signals are coupled capacitively through the skin, intervals 64 and 66 are each about 33 microseconds, intervals 68 and 70 each about 1 millisecond, intervals 72a and 72b each about 50 milliseconds, and intervals 74a and 74b each about 17 milliseconds. Alternate repetition of intervals 64 and 66 generates pulse bursts 80, each of the length of interval 68, each followed by a quiet interval of length 70 in which the signal remains substantially at level 60. Alternate repetition of intervals 68 and 70 then generates pulse burst groups 82, each of the length of interval 72a or 72b, each followed by a quiet interval of length 74a or 74b in which the signal remains substantially at level 60. Pulse burst groups 82 alternate in polarity, a group with peak level 62a, length 72a and followed by a quiet interval 74a alternating with a group with peak level 62b, length 72b and followed by a quiet interval 74b. Since lengths 72a and 72b are equal, and since all shorter intervals 64, 66, 68 and 70 are the same in all pulse burst groups, the resulting signal 56 has zero net charge (no D.C. component) over a full cycle of intervals 72a, 74a, 72b and 74b and has a duty cycle of about 37.5%.

In addition to stimulating bone fracture healing and relieving psychological conditions, electrical stimulation is also widely used in tissue healing applications. U.S. Pat. No. 5,974,342 issued in the name of Petrofsky discloses a microprocessor-controlled apparatus for treating injured tissue, tendon, or muscle by applying a therapeutic current. The apparatus has several channels that provide biphasic constant voltage or current, including a 100-300 microsecond positive phase, a 200-750 microsecond inter-phase, and a 100-300 microsecond negative phase occurring once every 12.5-25 milliseconds.

U.S. Pat. No. 5,723,001 issued in the name of Pilla, et al. discloses an apparatus for therapeutically treating human body tissue with pulsed radiofrequency electromagnetic radiation. The apparatus generates bursts of pulses having a frequency of 1-100 MHz, with 100-100,000 pulses per burst, and a burst repetition rate of 0.01-1000 Hz. The pulse envelope can be regular, irregular, or random.

U.S. Pat. No. 5,117,826 issued in the name of Bartelt, et al. discloses an apparatus and method for combined nerve fiber and body tissue stimulation. The apparatus generates biphasic pulse pairs for nerve fiber stimulation, and a net D.C. stimulus for body tissue treatment (provided by biphasic pulse trains having a greater number of negative than positive pulses). U.S. Pat. No. 4,895,154 also issued in the name of Bartelt, et al. describes a device for stimulating enhanced healing of soft tissue wounds that includes a plurality of signal generators for generating output pulses. The intensity, polarity, and rate of the output pulses can be varied via a series of control knobs or switches on the front panel of the device.

U.S. Pat. No. 5,018,525 issued in the name of Gu, et al. describes an apparatus that generates a pulse train made up of bursts having the same width, where each burst is made up of a plurality of pulses of a specific frequency. The number of pulses varies from one burst to the next; the frequency of the pulses in each burst varies from one burst to the next corresponding to the variation in the number of pulses in each burst. The pulses have a frequency of 230-280 KHz; the duty cycle of the bursts is between 0.33% and 5.0%.

U.S. Pat. No. 5,109,847 issued in the name of Liss, et al. relates to a portable, non-invasive electronic apparatus which generates a specifically contoured constant current and current-limited waveform including a carrier frequency with at least two low-frequency modulations. The carrier frequency is between 1-100,000 KHz; square-wave or rectangular-wave modulating frequencies are between 0.01-199 KHz and 0.1-100 KHz. Duty cycles may vary, but are typically 50%, 50%, and 75% for the three waveforms with the frequency noted above.

U.S. Pat. No. 4,612,934 issued in the name of Borkan describes a tissue stimulator that includes an implantable, subcutaneous receiver and implantable electrodes. The receiver can be noninvasively programmed after implantation to stimulate different electrodes or change stimulation parameters (polarity and pulse parameters) in order to achieve the desired response; the programming data is transmitted in the form of a modulated signal on a carrier wave. The programmed stimulus can be modified in response to measured physiological parameters and electrode impedance.

U.S. Pat. No. 4,255,790 issued in the name of Hondeghem describes a programmable pulse generating system where the time periods and sub-intervals of the output pulses are defined by signals from a fundamental clock frequency generation circuit, plus a pair of parallel sets of frequency division circuits connected to that circuit. The time periods, sub-intervals, and output waveforms are variable.

U.S. Pat. No. 3,946,745 issued in the name of Hsiang-Lai, et al. provides an apparatus for generating positive and negative electric pulses for therapeutic purposes. The apparatus generates a signal consisting of successive pairs of pulses, where the pulses of each pair are of opposite polarities. The amplitude, duration, the interval between the pulses of each pair, and the interval between successive pairs of pulses are independently variable.

U.S. Pat. No. 3,589,370 issued in the name of McDonald shows an electronic muscle stimulator which produces bursts of bidirectional pulses by applying unidirectional pulses to a suitable transformer.

U.S. Pat. No. 3,294,092 issued in the name of Landauer discloses an apparatus that produces electrical currents for counteracting muscle atrophy, defects due to poor nutrition, removing exudates, and minimizing the formation of adhesions. The amplitude of the output signals is variable.

U.S. Pat. Nos. 5,217,009; 5,413,596; 6,011,994; 6,321,119; 6,535,767 all issued in the name of Kronberg, and WIPO Publication No. WO 03015866 published in the name of Kronberg (these patents and publication are hereby incorporated by reference) describe signal generators for biomedical applications. The generators produce pulsed signals having fixed and variable amplitude, fixed, variable, and swept frequencies, and (in some cases) optional D.C. biasing.

Units designed for use in transcutaneous electroneural stimulation (“TENS”) for pain relief are widely available. For example, U.S. Pat. No. 5,487,759 issued in the name of Bastyr, et al. discloses a battery-powered device that can be used with different types of support devices that hold the electrode pads in position. Keyed connectors provide a binary code that is used to determine what type of support device is being used for impedance matching and carrier frequency adjustment. The carrier frequency is about 2.5-3.0 KHz; the therapeutic frequency is typically on the order of 2-100 Hz.

U.S. Pat. No. 5,350,414 issued in the name of Kolen provides a device where the carrier pulse frequency, modulation pulse frequency, intensity, and frequency/amplitude modulation are controlled by a microprocessor. The device includes a pulse modulation scheme where the carrier frequency is matched to the electrode-tissue load at the treatment site to provide more efficient energy transfer.

U.S. Pat. No. 4,784,142 issued in the name of Liss, et al. discloses an electronic dental analgesia apparatus and method. The apparatus generates a output with relatively high frequency (12-20 KHz) pulses with nonsymmetrical low frequency (8-20 Hz) amplitude modulation.

U.S. Pat. No. 5,063,929 issued in the name of Bartelt, et al. describes a microprocessor-controlled device that generates biphasic constant-current output pulses. The stimulus intensity can be varied by the user.

U.S. Pat. No. 4,938,223 issued in the name of Charters, et al. provides a device with an output signal consisting of bursts of stimuli with waxing and waning amplitudes, where the amplitude of each stimulus is a fixed percentage of the amplitude of the burst. The signal is amplitude-modulated to help prevent the adaptation response in patients.

U.S. Pat. No. 4,541,432 issued in the name of Molina-Negro, et al. discloses an electric nerve stimulation device for pain relief. The device produces a bipolar rectangular signal with a preselected repetition rate and width for a first time period. Then, a rectangular signal is generated at a pseudo-random rate for a second time period, and delivery of the signal is inhibited for a third, pseudo-random period of time. This protocol is said to substantially eliminate adaptation of nerve cells to the stimulation.

U.S. Pat. No. 4,431,000 issued in the name of Butler, et al. shows a transcutaneous nerve stimulator for treating aphasias and other neurologically-based speech and language impairments. The device uses a pseudorandom pulse generator to produce an irregular pulse train composed of trapezoidal, monophasic pulses which mimic typical physiological wave forms (such as the brain alpha rhythm). A series of such pulses has a zero D.C. level; a current source in the device reduces the effects of variables such as skin resistance.

U.S. Pat. No. 4,340,063 issued in the name of Maurer discloses a stimulation device which can be implanted or applied to the body surface. The amplitude of the pulse decreases with a degradation in pulse width along a curve defined by a hyperbolic strength-duration curve. This is said to result in proportionately greater recruitment of nerve fibers due to the nonlinear relationship between pulse width and threshold.

U.S. Pat. No. 4,338,945 issued in the name of Kosugi, et al. discloses a system operable to generate pulses that fluctuate in accordance with the 1/f rule. That is, the spectral density of the fluctuation varies inversely with the frequency: pleasant stimuli often have stochastic fluctuations governed by this rule. The system produces an irregular pulse train said to promote patient comfort during the stimulation.

Signal generators are also used in hearing prostheses. For example, U.S. Pat. No. 4,947,844 issued in the name of McDermott describes a receiver/stimulator that generates a series of short spaced current pulses, with between-pulse intervals of zero current having a duration longer than that of each spaced pulse. The waveform of the stimulus current includes a series of these spaced pulses of one polarity followed by an equal number of spaced pulses of opposite polarity so that the sum of electrical charge transferred through the electrodes is approximately zero.

U.S. Pat. No. 4,754,759 issued in the name of Allocca describes a neural conduction accelerator for generating a train of “staircase-shaped” pulses whose peak negative amplitude is two-thirds of the peak positive amplitude. The accelerator design is based on Fourier analysis of nerve action potentials; the output frequency can be varied between 1-1000 Hz.

U.S. Pat. No. 4,592,359 issued in the name of Galbraith describes a multi-channel implantable neural stimulator wherein each data channel is adapted to carry information in monopolar, bipolar, or analog form. The device includes charge balance switches designed to recover residual charge when the current sources are turned off (electrode damage and bone growth are said to be prevented by not passing D.C. current or charge).

Despite the great healing potential provided by the devices described above, traditional Western medicine has accepted electrotherapeutic treatment only grudgingly, and to date it is used only rarely. This seems to be a legacy from early beliefs that signals would need to have high local intensities to be effective. Most electrotherapeutic devices now available rely either on direct implantation of electrodes or entire electronic packages, or on inductive coupling through the skin using coils which generate time-varying magnetic fields, thereby inducing weak eddy currents within body tissues. The need for surgery and biocompatible materials in the one case, and excessive circuit complexity and input power in the other, has kept the price of most such apparatus (apart from TENS devices) relatively high, and has also restricted its application to highly trained personnel.

There remains a need for a versatile, cost-effective system that can be used to provide bioelectric stimulation in a wide range of applications, including healing acceleration and pain relief. There is also need in the art for a bioelectric stimulation system that is: power efficient, capable of being powered by safe, low-voltage batteries, and can reduce the likelihood of a shock hazard.

### SUMMARY OF THE INVENTION

An apparatus and method can generate an electrical signal for use in biomedical applications. The signal can be comprised of a control signal S_{C} representing the desired envelope of the final signal and switching among logic levels including zero (for quiet intervals) and one or more nonzero values (for intervals containing pulses) preferably including at least one pair of equal and opposite values L₁ and L₂. These may be combined on a single control line if desired, but in general it is more practical to let a plurality of lines carry different portions of the signal. For example, one line can carry a logic “1” only when S_{C} is nonzero, while a second line can carry a logic “1” or “0” respectively indicating positive or negative polarity of S_{C} during said nonzero periods.

During nonzero periods of S_{C}, a pulse oscillator can generate a train of pulses of desired length and with intervals of desired length between them. During periods when S_{C} equals zero, the oscillator can be disabled. Because no pulses are generated while S_{C} equals zero, the duty cycle is simply that percentage of the time when S_{C} is nonzero.

The pulses can then be amplified, attenuated, and/or switched in polarity to conform to the envelope specified by S_{C}. The pulses may then undergo further processing such as wave shaping or elimination of unwanted frequency components, and are then presented as an output in the form of a conductive device placed in contact with living tissue in order to provide bioelectric stimulation. Such conductive devices may include, but are not limited to, skin-contact electrodes, conductive wound dressings, conductive devices such as metal bone fixation pins or electrically-conductive catheters which have already been implanted for other purposes, or bodies of conductive liquid in contact with the skin or other tissues. Such conductive devices can provide a wide range of flexibility to suit individual cases.

An apparatus according to at least one embodiment can be lightweight, compact, self-contained, cost-effective to manufacture and maintain, convenient to carry or wear for extended periods, safe for unsupervised home use without the need for special training, and able to generate a signal as described above and deliver it efficiently to the body. Since only low voltages and currents are used, such an apparatus may not pose a shock hazard even in case of malfunction. Power can be conveniently furnished by compact and inexpensive batteries, needing replacement only once in several weeks of use.

The apparatus may be used to provide in vivo, customizable electrotherapeutic treatment for human and animal patients, including but not necessarily limited to healing acceleration, relief of acute or chronic pain, relief of swelling and/or inflammation, and when applied transcranially, relief of anxiety, depression, insomnia and related conditions. Since isolated cells or tissue cultures can also be affected by electrotherapeutic waveforms, the apparatus may also be used for in vitro applications.

The technique of generating the output signal is yet one advantageous feature. Conventional devices typically employ a “carrier” or continuously generated stream of short pulses which is then “modulated” by multiplication by the control signal S_{C}. In other words, the duty cycle of pulse generation is 100% even though the output duty cycle may be much less. This is wasteful of power, and various mechanisms have been employed to offset this waste.

In at least some of the present embodiments, however, since the short pulses (corresponding roughly to the carrier in the conventional devices) can be generated only when needed, that is with a duty cycle which matches that of the output, this waste of power can be substantially eliminated. In other words, a suitably designed and constructed pulse oscillator, when enabled by a control signal, can generate a pulsed signal and when not enabled by the control signal, the pulse oscillator can be completely shut off so that it consumes negligibly little power. By “negligibly little” is meant at least two and typically three or more orders of magnitude less power than when the same oscillator is enabled and running.

The apparatus for generating the signal is yet another advantageous feature. At least some embodiments can make it simple to generate any one or any combination of the signals described above by using a relatively simple circuit made of a varying number of inexpensive and widely-available, CMOS integrated circuit components.

With the substantial elimination of wasteful power generation associated with constant carrier signals, another advantageous feature can include the use of conventional, readily-available low-voltage batteries, such as alkaline or lithium batteries, as a power source for the system. While some embodiments may be used with A.C. (alternating current) power sources (with the addition of any suitable adapter), battery power not only reduces the size and weight of the system, but can also increase its safety and ease of use for a patient undergoing treatment. Alternatively, or additionally, some embodiments may employ other D.C. power sources. For example, some embodiments may employ rechargeable or reusable power sources such as ultra- or super-capacitors or fuel cells.

Typically, the batteries can be replaced at infrequent intervals (generally no more than once every few weeks, depending on the output signal and the particular application), simplifying patient compliance and reducing operating costs. With battery power, the possibility of electrical injuries is greatly reduced, since the generator is not connected to A.C. line current during use, does not produce high voltages (by the definition of standard EN60950), and does not generate frequencies likely to induce ventricular fibrillation. Only low power levels, such as are required to produce therapeutic effects, can be applied to the body. Thus, the generator cannot produce an electrical shock hazard even in the event of a malfunction: as a result, the invention is suitable for unsupervised home use.

Still another advantageous feature is versatility. The apparatus may be configured easily so as to produce an output waveform with selectable timing intervals, output voltage or current levels, and overall envelope, or to allow selection among a plurality of any of these, to address various physiological needs. Specifically, the output waveform can be based on a plurality of relatively long primary timing intervals T₁, T₂ and so forth, forming in succession a primary repeating cycle.

A plurality of shorter secondary timing intervals t₁, t₂ and so forth, into which at least one of the primary intervals is divided, can form in succession a secondary repeating cycle. This secondary repeating cycle can continue throughout the length of the one or more primary intervals, and can be generated only during one or more primary intervals, while at least one other of said primary intervals is not so divided. The secondary timing intervals and secondary repeating cycle are usually not generated during said primary intervals and are usually not divided. A plurality of constant voltage or current levels L₁, L₂ and so forth, one of which is presented to the output during each primary or secondary timing interval can be generated.

Other features and advantages of the various embodiments will be apparent to those skilled in the art from a careful reading of the detailed description presented below and the accompanying drawings and claims.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIGS. 1A-1C are waveform diagrams of typical waveforms used in stimulating bone fracture healing.

FIGS. 2A-2C are waveform diagrams of typical waveforms used in treating anxiety, depression, insomnia and related conditions when applied transcranially.

FIG. 3A is a schematic view of an electronic device according to one illustrated embodiment, configured to generate the signal of FIG. 1 and other signals.

FIG. 3B is a waveform diagram of waveforms generated by the electronic device shown in FIG. 3A according to one illustrated embodiment.

FIG. 4A is a schematic view of an electronic device according to another illustrated embodiment, configured to generate the signal of FIG. 2 and other signals.

FIG. 4B is a waveform diagram of waveforms generated by the electronic circuit shown in FIG. 4A according to one illustrated embodiment.

FIG. 5A is a schematic view of an electronic device according to another illustrated embodiment, configured to generate an alternative signal to those shown in FIGS. 1 and 2.

FIG. 5B is a waveform diagram of alternative waveforms to those shown in FIGS. 1 and 2 according to one illustrated embodiment.

FIG. 6 is a waveform diagram of waveforms similar to those in FIG. 5B, but deliberately unbalanced through pulse number modification according to one illustrated embodiment.

FIG. 7 is a waveform diagram of waveforms similar to those in FIG. 5, but deliberately unbalanced through suppression of one output polarity according to one illustrated embodiment.

FIG. 8 is a logical flow diagram of a process for providing complex bioelectric stimulation signals according to one illustrated embodiment.

### DETAILED DESCRIPTION OF EXEMPLARY EMBODIMENTS

In the following description, reference numerals are used to identify structural elements, portions of elements, surfaces or areas in the drawings, as such elements, portions, surfaces or areas may be further described or explained by the entire written specification. For consistency, whenever the same numeral is used in different drawings, it indicates the same element, portion, surface or area as when first used. Unless otherwise indicated, the drawings are intended to be read together with the specification, and are to be considered a portion of the entire written description of this invention. As used herein, the terms “horizontal,” “vertical,” “left,” right,” “up,” “down,” as well as adjectival and adverbial derivatives thereof, refer to the relative orientation of the illustrated structure as the particular drawing figure faces the reader.

Some embodiments are directed to an apparatus for use in providing bioelectric stimulation in a variety of applications. The apparatus generates a waveform having approximately rectangular or quasirectangular pulses repeated at a chosen frequency and in pulse bursts which recur at a lower chosen frequency and possess a chosen pattern over time. The characteristics of the waveform are variable to suit differing applications or target tissues to be treated, as will be described further below.

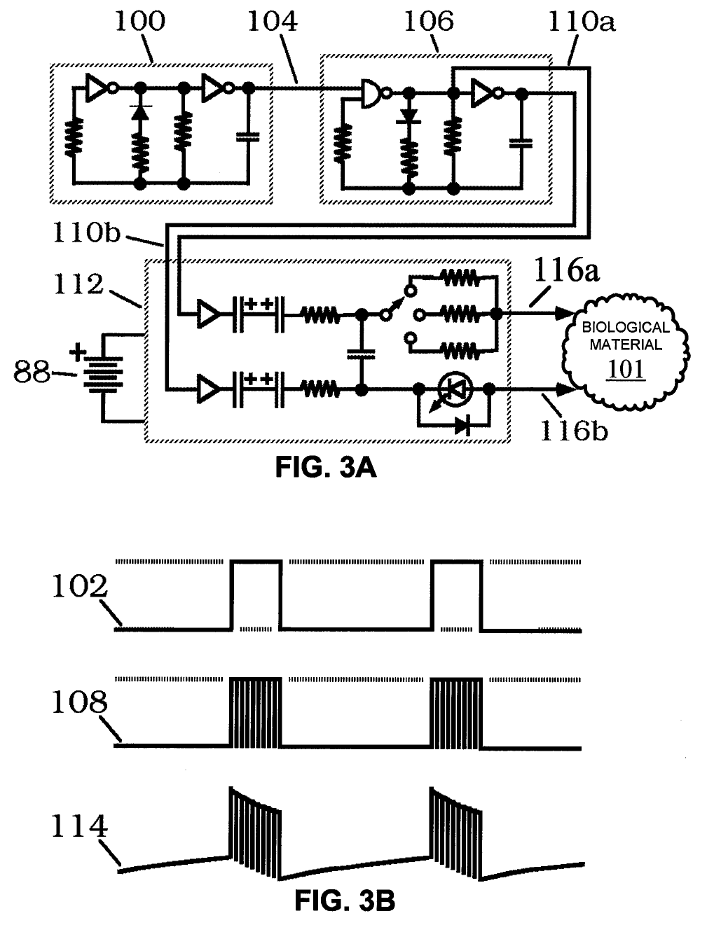

A first example of a signal generating device following the principles of the invention is described in U.S. Pat. No. 6,535,767 issued in the name of Kronberg, which is hereby incorporated by reference, and is shown simplified in FIG. 3A. Representative waveforms generated in its operation are illustrated in FIG. 3B. The device comprises a control oscillator 100, generating a control signal 102 on a control line 104.

A timing block particularly well-suited for generating asymmetric, repeating waveforms, such as control oscillator 100, is based on complementary metal-oxide-semiconductor (CMOS) logic. It is a little-known fact that a CMOS logic gate can function as either an analog or a digital device, or as both at once. This permits many signal generation and processing operations to be performed in a surprisingly effective and straightforward manner using CMOS logic gates with analog or mixed signals as inputs.

A self-starting, asymmetric CMOS oscillator 100 (technically, an astable multivibrator) based on this principle, comprises two inverting logic gates and a handful of passive components. The frequency of the oscillator depends on a time constant established by the capacitor and the three resistors. The polarity of the output waveform can be reversed if the polarity of diode 130 is reversed. Suitable values for the passive components may be found by first specifying a practical capacitor value typically in the range from about 100 picofarad to about 1 microfarad and then selecting the values of the resistors to establish the desired time constant and thus the operating frequency of the oscillator.

While the digital logic descriptions related to the various embodiments refer specifically to CMOS, other semiconductor technology may be used. Examples of other semiconductor technologies include, but are not limited to MOS, NMOS, PMOS, TTL, emerging transistor technology that introduces high-k dielectrics to replace silicon dioxide gate dielectrics, various other combinations of active devices such as FET's with or without passive devices such as resistors, and other like devices. One of ordinary skill in the art will appreciate that the use of CMOS technology may be advantageous because of the substantially zero static dissipation features of CMOS devices. That is, CMOS devices only dissipate significant power when switching. Semiconductor technology with reduced static power dissipation in general may be advantageous in designs related to the various embodiments. This quality may further benefit the total system power budget when the control signals have gated off the pulse generators, or in other power switching modes. With a reduced system power budget, simple battery power may be used and thus reduce any risk of shock that is often associated with A.C. power sources. A power supply 88 provided to power the signal generating system may be a battery of electrochemical cells, such as an alkaline battery, nickel cadmium, lithium, lithium ion, metal-acid, metal-base, electrolytic, or any other similar battery technology. Use of a battery as the power supply 88 may leverage the power saving features. Alternatively, the power supply may be any D.C. source or an A.C. to D.C. power adapter, such as a “wall wart” transformer or an automotive lighter/accessory power adapter for portable operation. For use in a medical application, such a source or adapter must provide sufficient electrical isolation according to applicable standards to ensure patient safety. The power supply 88 may also incorporate a charging circuit (e.g., battery charging circuit) and/or a mechanism for testing and displaying the charge or operation time remaining in the power supply 88 if not connected to an outside source of A.C. or D.C. power. While the power supply 88 is illustrated in FIG. 3A only connected to circuit stage 112, it is understood that all other circuit blocks, such as 100 and 106 may also be powered by the power supply 88.

Signal 102 of FIG. 3B is propagated along control line 104 and comprises a regularly alternating succession of logic “1” and logic “0” intervals, where logic “1” and “0” here are roughly equal to the positive and negative supply voltages respectively. A logic “1” then enables a second oscillator 106. The second oscillator 106 may be a circuit like control oscillator 100. The second oscillator 106 may be called the pulse oscillator, which in turn generates a differential output waveform 108 between lines 110a and 110b of FIG. 3A comprising pulse bursts during logic “1” periods of signal 102 and quiet periods during logic “0” periods of signal 102. The pulse oscillator 106 may be constructed just as the control oscillator 100 but with a shorter time constant for higher frequency operation. The logical NAND gate at the input of pulse oscillator 106 allows the gating control signal on line 104 to be combined (logically ANDed and then inverted) with the resulting signal being fed back within the oscillator itself to sustain oscillation. Alternatively, oscillator 106 could be constructed differently so as to be enabled by a logic “0” and disabled by a logic “1” through replacing this NAND gate with a NOR gate of equivalent characteristics.

Signal or waveform 108 illustrated in FIG. 3B and propagated along lines 110a and 110b of FIG. 3A can be further processed by components collectively indicated by 112, comprising logic gates, drivers or other amplifiers, resistors, capacitors and diodes. After processing by components 112 or state 112, signal 108 may become a differential output signal 114 of FIG. 3B between output conductive devices or conductive means 116a and 116b of FIG. 3A for bioelectric stimulation of biological material 101.

Such conductive devices/means 116 may include, but are not limited to, skin-contact electrodes, conductive wound dressings, conductive devices such as metal bone fixation pins or electrically-conductive catheters which have already been implanted for other purposes, other conductive devices such as wires or electro-acupuncture needles which have been inserted or implanted specifically for the purpose of bioelectric stimulation, or bodies of conductive liquid in contact with the skin or other tissues. Such conductive devices can provide a wide range of flexibility to suit individual cases.

The biological material 101 may include, but is not limited to, a human body, an animal (non-human) body, a complete organism, cells in culture, and tissue in culture.

In an exemplary and preferred embodiment, pulse oscillator 106 generates pulses of preferably 1 microsecond to 10 milliseconds in each polarity; more preferably of 10 to 1000 microseconds in each polarity; still more preferably with pulses of the two polarities having unequal lengths within the range from 10 to 1000 microseconds; and most preferably with pulses of one polarity lasting 10 to 100 microseconds while those of the other polarity last 100 to 1000 microseconds. However, other pulse lengths and combinations of polarities are not beyond the scope of this disclosure.

The pulses appear in bursts separated by quiet intervals, with the bursts and quiet intervals each preferably lasting between 100 microseconds and 10 seconds; more preferably between 1 millisecond and 1 second; still more preferably with said bursts having a different length from the quiet periods, each length lying between 1 millisecond and 1 second; and most preferably with the burst length lying between 1 and 20 milliseconds while the quiet period length lies between 5 and 200 milliseconds. Other burst lengths and quiet lengths and combinations thereof are not beyond the scope of this disclosure.

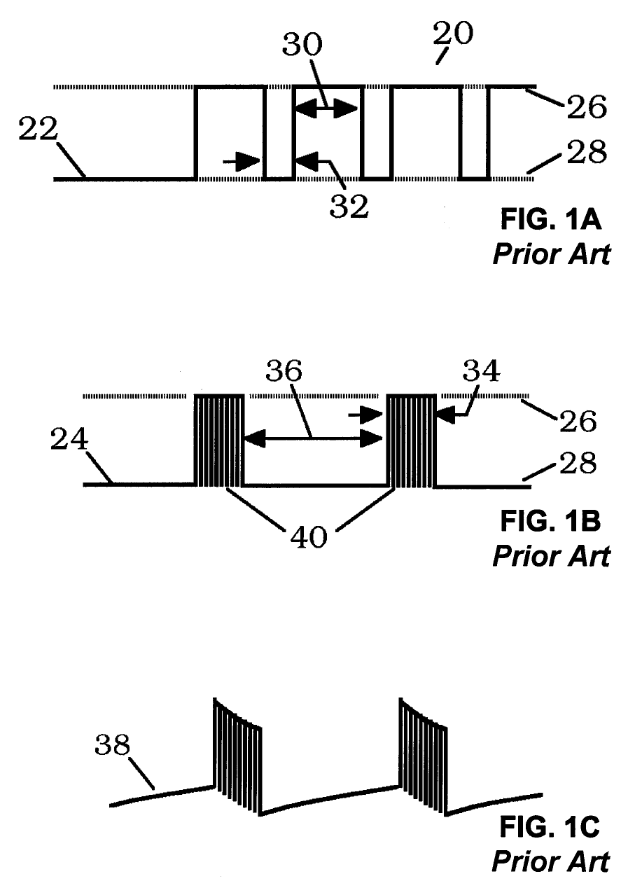

If values of 30 and 200 microseconds are assigned for the two pulse polarities, 5 milliseconds for the burst length and 60 milliseconds for the quiet period, waveform 108 illustrated in FIG. 3B becomes identical with waveform 24 of FIG. 1B, and output waveform 114 becomes identical with waveform 38 of FIG. 1C. An embodiment suitable for generating these signals is described in U.S. Pat. No. 6,011,994, which is here incorporated by reference. The waveform 114 of FIG. 3B is useful in bone fracture healing applications.

The short bursts of pulses illustrated in waveform 108 are only generated when the pulse oscillator 106 is enabled by the control signal 104 illustrated by waveform 102. In comparison with always generating the smaller pulses, there can be substantial reduction in the waste of power. This reduction in system power consumption can allow for the use of safe and simple battery power. Using battery power may also reduce the risk of shock hazard compared to conventional devices which may use A.C. power sources.

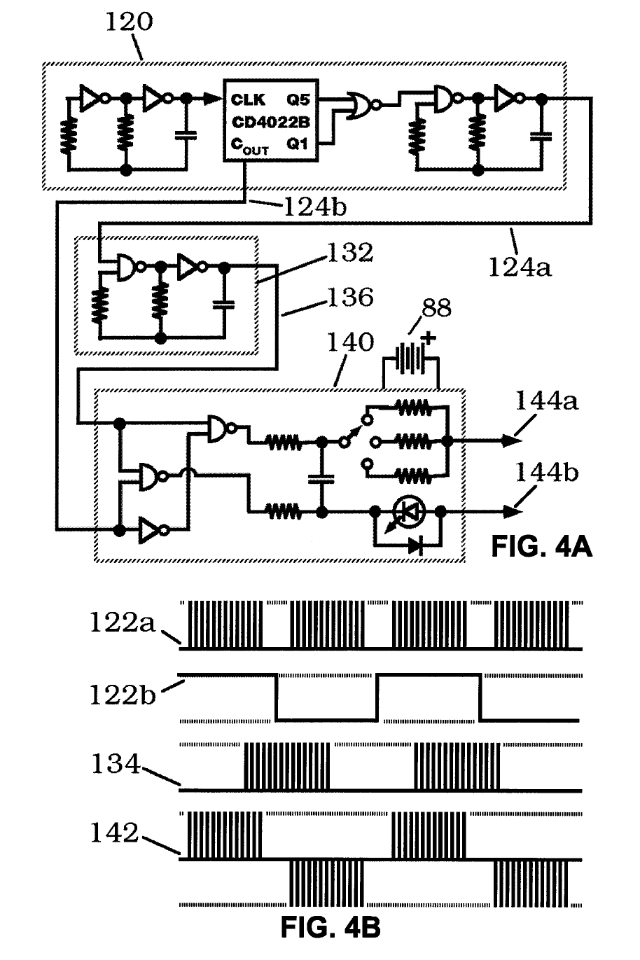

A second embodiment of a signal-generating device is illustrated in FIG. 4A. Representative waveforms provided by the device of FIG. 4A are illustrated in FIG. 4B. The device comprises a control oscillator 120. The control oscillator 120 outputs two control lines 124a and 124b.

Control line 124a carries a signal as illustrated by waveform 122a in FIG. 4B. This signal comprises a series of pulses that can be used to activate pulse oscillator 132 thereby generating a series of pulse bursts on line 136. The pulse bursts on line 136 are similar to those as illustrated by waveform 134 in FIG. 4B. When there is a logic “0” signal on control line 124a, pulse oscillator 132 is disabled and generates no output. However when there is a logic “1” signal on control line 124a, pulse oscillator 132 is activated to generate pulses.

Control line 124b has a lower frequency signal than control line 124a. The signal of control line 124b is similar to that illustrated by waveform 122b in FIG. 4B. Control signal or waveform 122b comprises alternating values of logic “1” and logic “0” that relate to the groups of enabling pulses in control signal 122a. That is, control signal 122b will be a logic “1” for one group of pulses in control signal 122a, but then control signal 122b will be a logic “0” for next group of pulses in control signal 122a, then control signal 122b will be a logic “1” for next group of pulses, and so on. Control signal 122b acts upon the components collectively indicated by circuit stage 140.

Circuit stage 140, provides a differential output signal between output connectors or treatment electrodes 144a and 144b. This output signal can be used for bioelectric stimulation. Circuit stage 140 provides an output signal similar to that illustrated by waveform 142. Control signal 122b causes circuit stage 140 to invert every other group of pulses from waveform 134.

In another preferred yet exemplary embodiment, pulse oscillator 132 generates pulses of preferably 1 microsecond to 10 milliseconds in each polarity; more preferably of 5 to 1000 microseconds in each polarity; still more preferably with pulses of the two polarities having equal lengths within the range from 5 to 1000 microseconds; and most preferably with pulses of each polarity lasting 10 to 100 microseconds. However, other pulse lengths and combinations of polarities are not beyond the scope of the subject embodiments.

The pulses appear in short bursts separated by short quiet intervals. In the second preferred embodiment, the short bursts and short quiet intervals each preferably last between 10 microseconds and 100 milliseconds; more preferably between 100 microseconds and 10 milliseconds; still more preferably with said short bursts and short quiet intervals having equal lengths within the range from 100 microseconds to 10 milliseconds; and most preferably with the short bursts and short quiet intervals each lasting from 500 microseconds to 2 milliseconds. Other burst lengths and quiet lengths and combinations thereof are not beyond the scope of the subject embodiments.

The short bursts and short quiet periods in turn alternate in burst groups, which are separated by longer quiet periods. In another preferred yet exemplary embodiment, the burst groups and longer quiet intervals each preferably last between 1 millisecond and 1 second; more preferably between 5 and 200 milliseconds; still more preferably with said burst groups having a different length from the longer quiet periods, each length lying between 5 and 200 milliseconds; and most preferably with the burst group length lying between 30 and 200 milliseconds while the longer quiet period length lies between 5 and 30 milliseconds. The burst groups alternate in polarity so that the total signal carries no net charge or D.C. component.

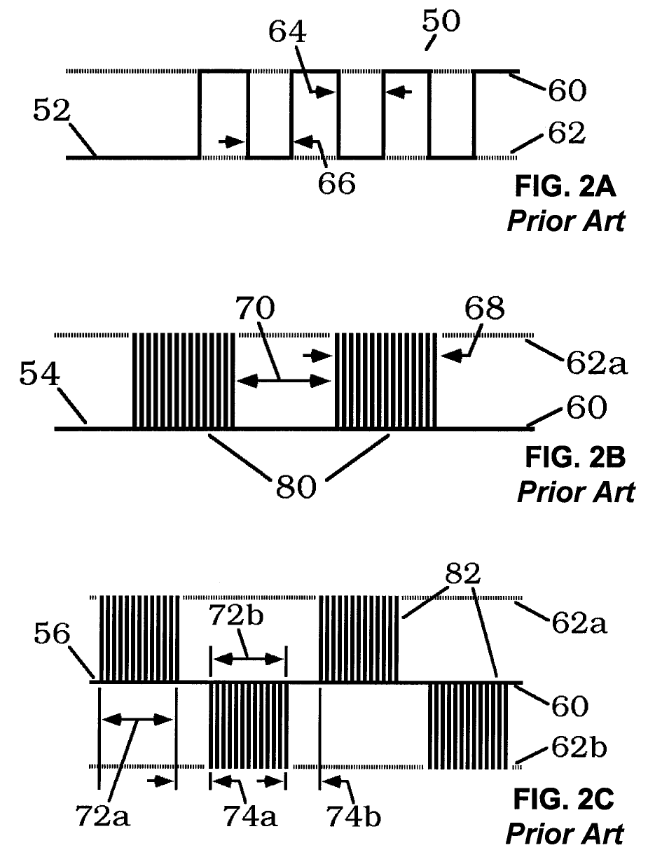

If assigned lengths of about 30 microseconds for the pulses in each polarity, about 1 millisecond each for the short pulse bursts and short quiet periods, about 50 milliseconds for the longer pulse burst and about 17 milliseconds for the longer quiet period, waveform 134 of FIG. 4B becomes identical with waveform 54 of FIG. 2B; waveform 122a of FIG. 4B becomes identical with waveform 54 of FIG. 2B; and output waveform 142 of FIG. 4B becomes identical with waveform 56 of FIG. 2C. Individual pulses in signal 142 of FIG. 4B are not shown; only pulse bursts and pulse burst groups are visible.

The output waveform 142 of FIG. 4B may be provided by conventional devices described in the background, but this waveform 142 may be generated in a more efficient manner by the various embodiments taught herein which do not require the continuous generation of a carrier signal. Since the short bursts of pulses illustrated in waveform 108 are only generated when the pulse oscillator 106 is enabled by the control signal 104 illustrated by waveform 102, there can substantial reduction of wasted power. This reduction in system power consumption can allow for the use of safe and simple battery power. Using battery power may also reduce the risk of shock hazard compared to prior art devices which may use A.C. power sources. The output waveform 142 of FIG. 4B may be useful in pain relief applications and when applied to the head, it may be useful for relief of depression, anxiety, and insomnia.

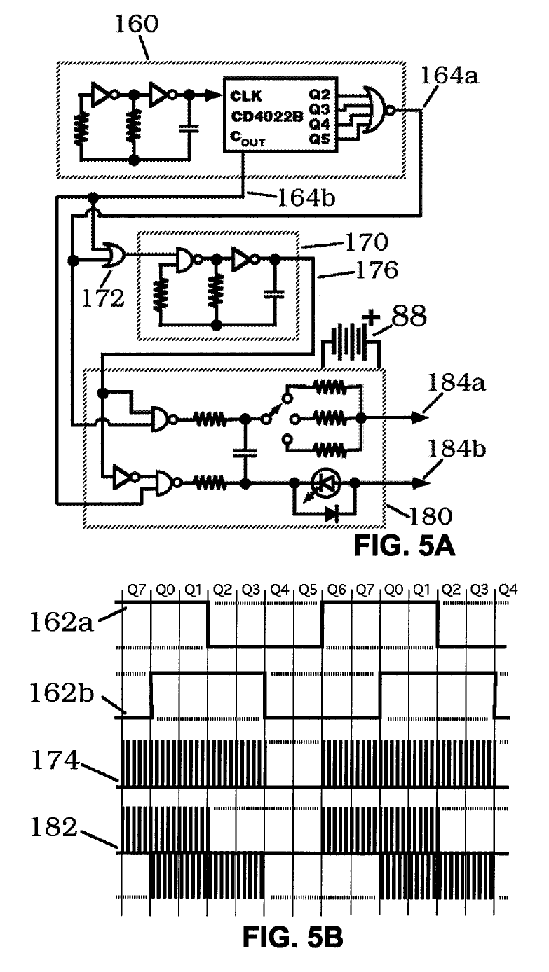

Yet another embodiment of a signal-generating device is illustrated in FIG. 5A. Representative waveforms generated by the device such as that in FIG. 5A are illustrated in FIG. 5B. The device comprises a control oscillator 160. The control oscillator 160 generates dual control signals on lines 164a and 164b respectively. The control signal on line 164a can be similar to that illustrated by waveform 162a in FIG. 5B. The control signal on line 164b can be similar to that illustrated by waveform 162b in FIG. 5B.

A logic “1” on line 164a indicates the presence of positive-polarity pulses in the output while a logic “1” on line 164b indicates the presence of negative-polarity pulses in the output. This scheme permits any of four different conditions: logic “0” on both lines causing a quiet output at zero voltage; logic “1” on line 164a only causing an alternation between zero and positive output; logic “1” on line 164b only causing an alternation between zero and negative output; and logic “1” on both control lines at once causing an alternation between positive and negative output levels.

All such alternations take place at the frequency of a pulse oscillator 170. The pulse oscillator 170 is enabled through logic gate 172 when either line 164a or 164b (or both) carries a logic “1” but disabled when both carry “0.” Oscillator 170 produces an output signal on line 176. The output signal on line 176 can be similar to that illustrated by waveform 174 of FIG. 5B. Components collectively indicated by 180 then process the output signal in the manner previously described, yielding a differential signal between output connectors or treatment electrodes 184a and 184b. This output signal can be similar to that illustrated by waveform 182 in FIG. 5B. This output signal can be used for bioelectric stimulation. Specifically, the output signal can be used to relieve pain in humans and other like applications.

Additional information on circuit devices which may potentially be used, and additional modes for carrying out the various embodiments according to the principles here described, may be found in U.S. Pat. Nos. 5,217,009; 5,413,596; 6,011,994; 6,321,119; 6,535,767; 7,117,034, and Reissue application Ser. No. 11/084,870 filed on Mar. 18, 2005 (corresponding to U.S. Pat. No. 6,535,767).

A waveform of the general type described above will inherently be charge-balanced—that is, the output will show a net zero D.C. content—if the time average of positive and negative voltages or currents at the output, over the length of one primary cycle, is zero. This may be achieved in any of several ways. For example, the output may be passed through an output network which blocks D.C. In the device described in U.S. Pat. No. 6,535,767 and shown in simplified form in FIG. 3A, the capacitors forming a part of the output network of block 112 filter out any D.C. component present. Alternatively, the positive and negative signal intervals may be balanced so that approximately equal amounts of time are spent in each state, minimizing the D.C. content. This approach is taken in the devices shown in FIGS. 4 and 5.

In other applications, for instance in iontophoresis or in the acceleration of wound healing through cell galvanotaxis, it is desirable to introduce a controlled D.C. content superimposed on the principal, A.C. waveform. This may be done simply by unbalancing the time spent in positive and negative intervals, so that one polarity predominates, while eliminating any downstream components, such as series capacitors, which would block the desired D.C. signal content.

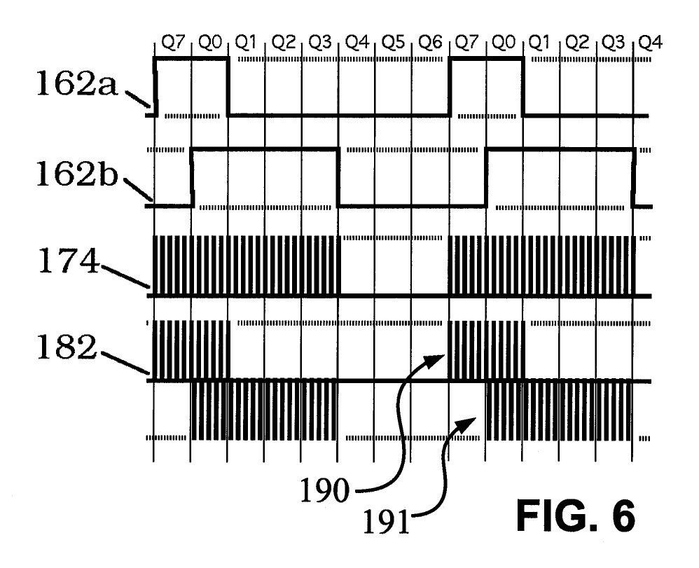

An example of an unbalanced waveform with a dominant polarity is illustrated in FIG. 6. This waveform may be generated, for example, by different inputs to the NOR gate producing signal 162a. Note that this waveform is simply the waveform which was previously illustrated in FIG. 5, but here made asymmetrical. That is, the output waveform 182 in FIG. 6 has more pulses of negative polarity 191 than it has pulses of positive polarity 190. For easy comparison, the identifying characters of FIG. 5 have been retained unchanged.

A close examination of the waveforms of FIG. 6 reveal that control signals 162a and 162b operate on the pulse oscillator that generates high density pulses 174 in a signal similar to that illustrated by waveform 174 in FIG. 5B. The output waveform 182 of FIG. 6 demonstrates an intentional charge imbalance as the signal is negative more than it is positive. That is, the output waveform 182 in FIG. 6 has more pulses of negative polarity 191 than it has pulses of positive polarity 190. The pulse bursts 190 are positive during the non-zero periods of waveform 162a and the pulse bursts 191 are negative during the slightly longer non-zero periods of waveform 162b.

In FIG. 6, the control signal waveform 162a has pulses that are only two clock cycles in duration, while the control signal waveform 162a in FIG. 5B has pulses that are four clock cycles in duration. Here, a clock cycle is defined as the time periods indicated as Q0-Q7 in both FIG. 5B and FIG. 6. This difference in control signal 162a between FIGS. 5B and 6 is further reflected in the positive polarity pulses 190 at the output signal 182 of FIG. 6.

While the waveform 182 in FIG. 5B is charge balanced in the steady state, the waveform 182 in FIG. 6 has fewer positive polarity pulses 190. This reduction in pulses is directly related to the reduction in the non-zero pulse width of signal 162a in FIG. 6. The rising edge from clock Q6 to Q7 of signal 162a in FIG. 6 enables the generation of positive polarity pulses 190 within waveform 182 at the same clock transition. The falling edge from clock Q0 to Q1 of signal 162a in FIG. 6 disables the generation of positive polarity pulses 190 within waveform 182 at the same clock transition.

Since the non-zero portion of control signal 162a controls the generation of pulse burst 190, their occurrence in time is substantially coincident. The same control relationship can be drawn between the control signal 162b and the negative polarity pulses 191 in waveform 182 of FIG. 6. Furthermore, the pulse generator output shown in waveform 174 of FIG. 6 is enabled by either control signal 162a or control signal 162b have a non-zero pulse present in FIG. 6.

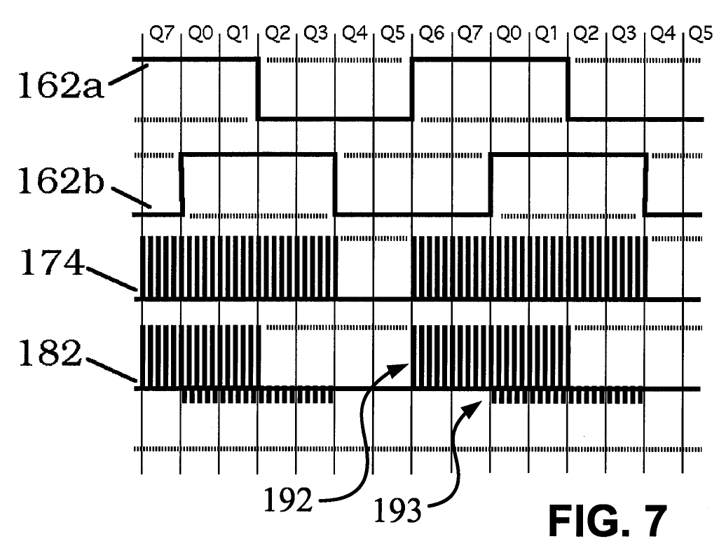

Alternatively, the waveform may be deliberately unbalanced by making the polarities asymmetrical around zero. For example, the waveform may be made unbalanced by substantially suppressing all pulses of one polarity as illustrated in FIG. 7. Here the positive pulses 192 are again as illustrated in FIG. 5B but negative pulses 193 also may be partially or entirely suppressed (not illustrated).

This result can be achieved, for example, by placing a Schottky or other type diode between outputs 184a and 184b. While the negative pulses 193 are illustrated as suppressed, the positive polarity pulses 192 may be suppressed in yet another embodiment.

Again for easy comparison, the identifying characters of FIG. 5 have been retained in FIG. 7 substantially unchanged. A close examination of the waveforms of FIG. 7 reveal that control signals 162a and 162b operate on the pulse oscillator that generates high density pulses 174 in a signal similar to that illustrated by waveform 174 in FIG. 7. However, the output waveform 182 of FIG. 7 demonstrates an intentional charge imbalance. That is, the negative polarity pulses 193 may be reduced in magnitude while the positive polarity pulses 192 remain substantially unchanged. The pulse bursts 192 are positive during the non-zero periods of waveform 162a and the pulse bursts are negative 193 (but of a reduced magnitude) during the non-zero periods of waveform 162b.

In FIG. 7, the control signal waveforms 162a and 162b are substantially identical to the control signals 162a, 162b illustrated in FIG. 5B. The difference between the resultant output signal 182 in FIG. 7 and the output signal 182 in FIG. 5B is the reduction in amplitude of the negative polarity pulses 193 illustrated in FIG. 7. Just as in FIG. 5B, the rising edge from clock Q7 to Q0 of signal 162b in FIG. 7 enables the generation of negative polarity pulses 193 within waveform 182 at the same clock transition.

Just as in FIG. 5B, the falling edge from clock Q3 to Q4 of signal 162b in FIG. 7 disables the generation of negative polarity pulses 193 within waveform 182 at the same clock transition. Since the non-zero portion of control signal 162b controls the generation of pulse burst 193, their occurrence in time is substantially coincident. The same control relationship can be drawn between the control signal 162a and the positive polarity pulses 192 in waveform 182 of FIG. 7. Again, the significant difference between the waveforms illustrated in FIG. 5B and those in FIG. 7, is the reduction in amplitude of the negative polarity pulses 193 in FIG. 7. This reduction may be used to intentionally generate an unbalanced charge at the output for use in certain biomedical treatment techniques.

The embodiments described above should not be interpreted as restricting the scope of the invention, since various embodiments may provide a maximum range of possible output signals, achievable by like means and using like circuitry, but not all necessarily having similar envelopes. For example, features of the output signal which could potentially be controlled by a suitably designed control signal S_{C} include not only pulse generation and polarity, but intensity, pulse timing, charge balance, and through proper manipulation of some combination of these, the emulation of specific, definable mathematical functions such as sine waves or their combination to create beat frequencies. Other mathematical functions include, but are not limited to the following: a constant value; a sine function; a sum of sine functions creating a beat frequency; a constant value which is intermittent with time forming a square or rectangular wave; an arithmetic combination, such as the sum, product or ratio, of two or more of the functions or function types just mentioned; or randomness.

Control signals S_{C} described above have all been periodic, repeating with time, but aperiodic signals, such as randomly generated series of control pulses, could also be used. Suitable random series generation techniques may be applied by those of skill in the art of circuit design and waveform analysis.

Many additional waveforms and means of generating them should now be apparent to anyone skilled in the art of circuit design or waveform analysis.

For example, the control signal has two possible states comprising turning said pulse oscillator on and off. Turning the oscillator on or off may have a pattern in time. The pattern may be a regularly alternating succession of “on” and “off” pulses. The “on” pulses that enable the pulse oscillator recur regularly with time. For instance, the “on” pulses that enable the pulse oscillator recur in a regularly repeating pattern with time. The pattern may include groups of “on” pulses. The groups of “on” pulses may be separated by quiet periods without said “on” pulses. In some embodiments, the pattern in time may be random.

Also for example, the pulse oscillator may generate a pulsed signal comprising pulses of 1 microsecond to 10 milliseconds in each polarity. The pulse oscillator may generate a pulsed signal comprising pulses of 10 to 1000 microseconds in each polarity. The pulse oscillator may generate a pulsed signal comprising pulses of the two polarities having unequal lengths within the range from 10 to 1000 microseconds. The pulse oscillator may generate a pulsed signal comprising pulses of one polarity lasting 10 to 100 microseconds while those of the other polarity last 100 to 1000 microseconds. The pulse oscillator may generate a pulsed signal comprising pulses of one polarity lasting approximately 30 microseconds, while those in the other polarity last approximately 200 microseconds. The pulsed signal may comprise bursts separated by quiet intervals, and said bursts and quiet intervals each last between approximately 100 microseconds and 10 seconds. The bursts and quiet intervals each last between approximately 1 millisecond and 1 second. The bursts may have a different length from the quiet periods, each burst length may, for example be between approximately 1 millisecond and 1 second. For example, the burst length may be between approximately 1 and 20 milliseconds while the quiet period has a duration of between approximately 5 and 200 milliseconds. The length of the burst duration may be approximately five to ten milliseconds while the burst and quiet period together are repeated at approximately 15 Hz.

The pulse oscillator may generate a pulsed signal comprising pulses of 5 to 1000 microseconds in each polarity. The pulse oscillator may generate a pulsed signal comprising pulses with polarities having equal lengths within the range from approximately 5 to 1000 microseconds. The equal lengths may be between approximately 10 to 100 microseconds, for instance the equal lengths may be approximately 30 microseconds. The pulsed signal may includes pulses with short bursts separated by short quiet periods, the short bursts and short quiet periods grouped in turn into burst groups separated by longer quiet periods. Such short bursts and short quiet intervals may each last between approximately 10 microseconds and 100 milliseconds. For instance, the short bursts and short quiet intervals may each last between approximately 100 microseconds and 10 milliseconds. The short bursts and short quiet intervals may, for example, have equal lengths within the range from 100 microseconds to 10 milliseconds. The short bursts and short quiet intervals each last from 500 microseconds to 2 milliseconds. The short bursts and short quiet intervals may each last approximately 1 millisecond. The burst groups and longer quiet intervals may each last between approximately 1 millisecond and 1 second. The burst groups and longer quiet intervals may each last between approximately 5 and 200 milliseconds. The burst groups may have a different length from said longer quiet periods, each said length comprising between approximately 5 and 200 milliseconds. The burst group length may, for example be between approximately 30 and 100 milliseconds, for instance approximately 50 milliseconds. The longer quiet period length may be between approximately 5 and 30 milliseconds. The longer quiet period length may, for example be approximately 17 milliseconds. The burst groups may alternate in polarity so that the total signal carries does not comprise a net charge or D.C. component.

The promotion of therapeutic effects in the biological material 101 and conditions believed to be treatable with waveforms, such as those described above produced by the various embodiments, may include, but are not necessarily limited to, the following: bone fractures, osteoporosis, acute pain, chronic pain, swelling, simple inflammation, and inflammatory disorders such as tendonitis (including carpal tunnel syndrome and other repetitive stress injuries), osteoarthritis and rheumatoid arthritis. Accelerated healing of wounds, involving a variety of tissue types and resulting either from trauma or from degenerative conditions such as diabetes, may also be promoted with the output waveforms.

Skin ulcers, such as diabetic or decubitus ulcers, may respond well to the output waveforms. Nerve function may be improved or restored, for instance following trauma or in cases of diabetic neuropathy. Applied transcranially, the output signals described herein may relieve anxiety, depression, insomnia and related conditions. However, it should be understood that no one set of timing intervals, output intensity, polarity, or polarity reversal is necessarily useful for treating all (or even most) of these conditions.

It is believed that appropriate voltage/current levels and timing intervals may be used to treat a wider variety of conditions whose etiology involves improper rates or imbalances in cell, organ or whole-body metabolism, secretion or replication, or which can be relieved by suitably modifying these factors. Thus, it should be understood that the optimum waveform characteristics for each particular application are best found with a modest combination of observation and without undue experimentation.

An apparatus/system according to the various embodiments may be used to promote one or more therapeutic effects, such as providing electrotherapeutic treatment for human and animal patients, including but not limited to, healing acceleration, relief of acute or chronic pain, and relief of swelling and/or inflammation. However, the apparatus need not be confined to use with intact organisms, since isolated cells or tissue cultures can also be affected by electrotherapeutic waveforms (appropriate electrical stimuli have been observed to modify the rates of cell metabolism, secretion, and replication). Isolated skin cells, for example, might be treated with selected waveforms in an appropriate medium to increase cell proliferation and differentiation in the preparation of tissue-cultured, autogenous skin-graft material.

As another example of promoting a therapeutic effect in biological material 101, the growth of bacteria or other organisms genetically engineered to produce a desirable product, such as human insulin, may be accelerated, or their secretion of the desired product increased, by treatment with a suitable waveform. As yet another example of a therapeutic effect, human cells or tissues in culture might be treated to increase proliferation, speed the development of more mature tissue structure, or enhance the secretion of a desired substance or combination of substances, such as transforming growth factor beta, insulin-like growth factor 1 (IGF-1), and other related growth factors in bone material meant for grafting.

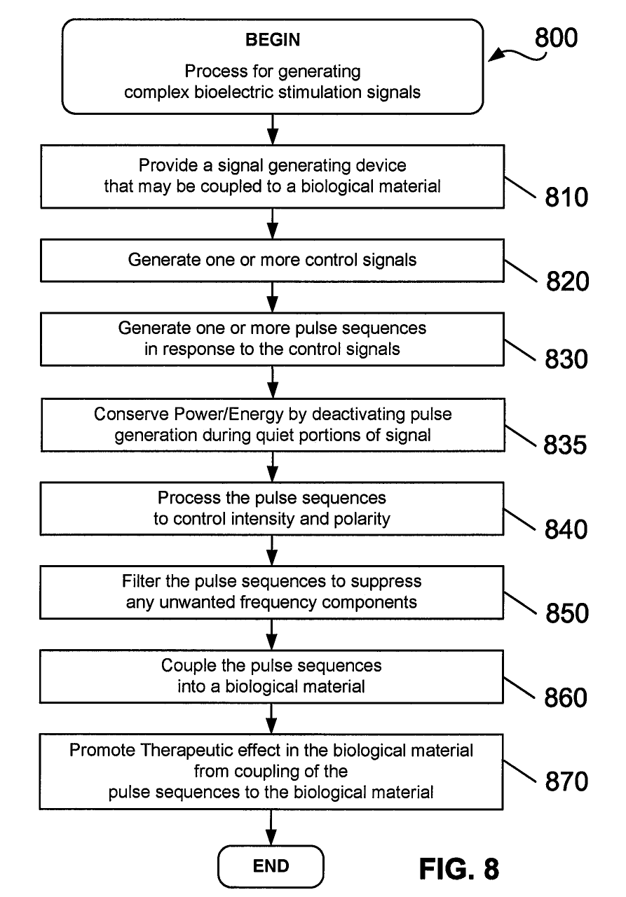

FIG. 8 shows a logical flow diagram 800 of a process for providing complex bioelectric stimulation signals according to one exemplary embodiment. Certain acts in the processes or process flow described in all of the logic flow diagrams referred to below must naturally precede others to function as described. However, the various embodiments are not limited to the order of the acts described if such order or sequence does not alter the functionality of one or more of the embodiments. That is, it is recognized that some acts may be performed before, after, or in parallel with other acts.

The process 800 for generating complex bioelectric stimulation signals may begin at 810 where a signal generating device is provided that may be coupled to a biological material 101. Such a device can be those illustrated in FIGS. 3A, 4A or 5A.

At 820, one or more control signals are generated to control the generation of the complex signals. These control signals may determine the various parameters associated with the complex stimulation signals, such as duty cycle, duration, timing, delay periods, amplitudes, phases, polarities, frequency content, D.C. offset, and charge unbalance.

At 830, one or more pulse sequences are generated in response to the control signals. The envelopes, bursts, group bursts, delays between bursts, delays between group bursts, and other timing associated with these pulse sequences may be controlled by the control signals generated at 820.

At 835, power/energy can be conserved by deactivating pulse generation during quiet portions of signal. That is, when the control signal generated at 820 indicates a quiet period without pulses, the pulse generation at 830 may be entirely disabled to conserve power. Such power efficiencies can allow the signal generating system to use less and/or smaller batteries and less power. Also, the battery supply for the system may last longer in a system that is more power efficient. A battery powered system may also be safer and can reduce any potential shock hazards compared to prior art devices which may be required to use A.C. power.

At 840, the pulse sequences can be processed to control the intensity and polarity of the pulses or bursts of pulses. This processing may be in response to one or more of the control signal generated at 820.

At 850, the pulse sequences may be filtered to suppress any unwanted frequency components. This filtering may be in response to one or more of the control signal generated at 820. The filtering of unwanted frequencies may include the suppression of an unwanted D.C. component. This may further comprise the addition of a D.C. component or a pulse with a desired D.C. offset. These D.C. additions may be operable to equalize charge balance or to intentionally offset the charge balance to a desired level and polarity.

At 860, the pulse sequences may be coupled into a biological material 101. The coupling of the signals may occur by any combination of leads, terminals, contacts, pads, electrodes, electromagnetic radiation, or other coupling mechanisms. The coupling may be transcutaneous, transcranial, in vivo, in vitro, or otherwise. The coupling may be to a cell, multiple cells, tissue, systems, limbs, organs, or to an organism as a whole, for example, a human or portion thereof.

At 870, a therapeutic effect in the biological material may be promoted from the coupling of the pulse sequences into the biological material 101. The complex stimulation signals, pulses, and pulse bursts coupled to the biological material 101 at 860 can interact with the electrical and electrochemical properties of the biological material 101 to deliver stimulation to the biological material 101. Example of such properties may be conductivity, capacitance, reactance, resistance, reactivity, ion concentration, lipid content, pH, moisture content, dielectric properties, time constants, and any combination or interaction thereof. While the process 800, or parts of the process 800, may certainly be carried out in a continuous or looping manner, the example may be said to terminate after 870 for non-limiting illustrative purposes.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art. The teachings provided herein of the various embodiments can be applied to other medical devices (e.g., therapeutic and/or diagnostic), not necessarily the exemplary bioelectric stimulation devices generally described above.

For instance, the foregoing detailed description has set forth various embodiments of the devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one embodiment, the present subject matter may be implemented via Application Specific Integrated Circuits (ASICs). However, those skilled in the art will recognize that the embodiments disclosed herein, in whole or in part, can be equivalently implemented in standard integrated circuits, as one or more computer programs running on one or more computers (e.g., as one or more programs running on one or more computer systems), as one or more programs running on one or more controllers (e.g., microcontrollers) as one or more programs running on one or more processors (e.g., microprocessors), as firmware, or as virtually any combination thereof, and that designing the circuitry and/or writing the code for the software and or firmware would be well within the skill of one of ordinary skill in the art in light of this disclosure.

In addition, those skilled in the art will appreciate that the mechanisms of taught herein are capable of being distributed as a program product in a variety of forms, and that an illustrative embodiment applies equally regardless of the particular type of signal bearing media used to actually carry out the distribution. Examples of signal bearing media include, but are not limited to, the following: recordable type media such as floppy disks, hard disk drives, flash or battery-backed static memory, CD ROMs, digital tape, and computer memory; and transmission type media such as digital and analog communication links using TDM or IP based communication links (e.g., packet links).

The various embodiments described above can be combined to provide further embodiments. To the extent that they are not inconsistent with the specific teachings and definitions herein, all of the U.S. patents, U.S. patent application publications, U.S. patent applications, foreign patents, foreign patent applications and non-patent publications referred to in this specification and/or listed in the Application Data Sheet, including but not limited to: U.S. Pat. No. 5,217,009 issued Jun. 8, 1993; U.S. Pat. No. 5,413,596 issued May 9, 1995; U.S. Pat. No. 6,011,994 issued Jan. 4, 2000; U.S. Pat. No. 6,321,119 issued Nov. 20, 2001; U.S. Pat. No. 6,535,767 issued Mar. 18, 2003; and U.S. Pat. No. 7,117,034 issued Oct. 3, 2006; and Reissue application Ser. No. 11/084,870 filed on Mar. 18, 2005 (corresponding to U.S. Pat. No. 6,535,767) all of which are incorporated herein by reference, in their entirety. Aspects of the embodiments can be modified, if necessary, to employ systems, circuits and concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A system for generating an electrical signal for use in biomedical applications, comprising:
means for generating a control signal having at least two states alternating in a pattern as a function of time, the pattern comprising a succession of “on” and “off” pulses which recur in a regularly repeating pattern with time;
a pulse oscillator which is enabled during the “on” pulses of the control signal and generates a pulsed signal, but is disabled and consumes negligibly little power during the “off” pulses of the control signal;
processing means for processing the pulsed signal, the processing means also performing at least one of controlling a signal intensity, inverting a portion of the pulsed signal, and suppressing at least one of direct current (D.C.) and frequency components of the pulsed signal, thereby creating an output signal; and
conductive means for conducting and applying the output signal to a biological material for promoting a therapeutic effect in the biological material.

2. The system of claim 1, in which the pattern as a function of time emulates one of the following mathematical functions: a constant value; a sine function; a sum of sine functions creating a beat frequency; a constant value which is intermittent with time forming a square or rectangular wave; an arithmetic combination, such as the sum, product or ratio, of two or more of the functions or function types; or randomness.

3. The system of claim 1, in which the control signal comprises in addition to the “on” and “off” pulses, one or more auxiliary signals that control at least one of a polarity; an intensity; a timing of pulses; or a charge balance of the output signal.

4. The system of claim 1, in which the pulse oscillator generates the pulsed signal such that the pulses alternate between two polarities and have equal pulse lengths, each of the pulse lengths lying in the range from 1 microsecond to 1000 milliseconds, inclusive.

5. The system of claim 1, in which the pulse oscillator generates the pulsed signal such that the pulses have unequal lengths in two polarities, the pulses of one polarity lasting 10 to 100 microseconds while the pulses of the other polarity lasting 100 to 1000 microseconds.

6. The system of claim 1, in which the pulse oscillator generates the pulsed signal such that the pulses are grouped into bursts separated by quiet periods.

7. The system of claim 1, in which the pulse oscillator generates the pulsed signal such that the pulses are grouped into a plurality of short bursts, pairs of the short bursts separated by a respective short quiet period, the short bursts and the short quiet periods grouped into a plurality of burst groups, pairs of the burst groups separated by a respective longer quiet period, the longer quiet periods longer in duration than the short quiet periods.

8. The system of claim 7, in which the short bursts and the short quiet periods each last between approximately 10 microseconds and 100 milliseconds while the burst groups and the longer quiet periods each last between approximately 5 and 200 milliseconds.

9. The system of claim 7, in which the short bursts and the short quiet periods each last between approximately 1 millisecond and 20 milliseconds while the short quiet periods and the longer periods each last between approximately 5 and 200 milliseconds.

10. The system of claim 7, in which the pulse oscillator generates the pulsed signal such that the pulses are of approximately 5 microseconds to 1000 microseconds of each polarity.

11. The system of claim 7, in which a second one of the burst groups in each pair of the burst groups is inverted relative to the first one of the burst groups in the pair, such that the pulsed signal does not comprise a cumulative net charge or D.C. component.

12. The system of claim 1, in which the conductive means comprises at least one of: skin-contact electrodes; a conductive wound dressing; a metal bone fixation pin; an electrically-conductive catheter; a conductive device, wire or electro-acupuncture needle inserted or implanted for the purpose of bioelectric stimulation; or a body of conductive liquid in contact with tissue.

13. The system of claim 1, in which the biological material comprises at least one of a human body, an animal body, a complete organism, cells in culture, or tissue in culture.

14. The system of claim 1, in which the therapeutic effect comprises at least one of the following: an increase in cell proliferation, cell differentiation, rate of organism growth, secretion of a desired product, or speed with which a tissue structure is developed; treatment of a wound, a bone fracture, osteoporosis, acute pain, swelling, an inflammatory disorder, a repetitive stress injury, osteoarthritis, and rheumatoid arthritis; accelerated healing of at least one wound; an improvement or restoration of nerve function; or relief of a psychological condition.

15. The system of claim 1, in which all power is supplied with one or more primary batteries comprising at least one of alkaline batteries, lithium batteries, rechargeable batteries, or a combination of disposable and rechargeable batteries.

16. A system for generating bioelectric stimulation signals comprising:
a controller that produces a control signal having at least two states;
an oscillator coupled to the controller that generates a pulsed signal in response to a first one of the states of the control signal, and that is turned off and consumes negligibly little power in response to a second one of the states of the control signal;
a processor coupled to receive the pulsed signal, and configured to suppress at least one of a direct current (D.C.) component and a frequency component of the pulsed signal, to produce an output signal; and
a conductive device coupled to the processor to transfer the output signal to a biological material to promote a therapeutic effect in the biological material.

17. The system of claim 16, further comprising a power source that includes a battery electrically coupled to supply power to the system.

18. The system of claim 16, wherein at least one of the controller and oscillator comprises a complementary metal-oxide-semiconductor (CMOS) circuit.

19. A system for generating an electrical signal for use in biomedical applications, comprising:
a controller configured to generate a control signal having at least two states alternating in a pattern as a function of time, the pattern comprising a succession of “on” and “off” pulses which recur in a regularly repeating pattern with time; the control signal further having one or more auxiliary signals to control at least one of a polarity, an intensity, a timing of pulses, or a charge balance of an output signal;
a pulse oscillator which is enabled during the “on” pulses of the control signal and generates a pulsed signal, but is disabled and consumes negligibly little power during the “off” pulses of the control signal;
a circuit coupled to receive the pulsed signal and configured to at least one of control a signal intensity of the pulsed signal, invert a portion of the pulsed signal, and suppress at least one of a direct current (D.C.) component and a frequency component of the pulsed signal, to produce the output signal having a pattern of intensity and polarity as functions of time which emulates one of the following mathematical functions: a constant value; a sine function; a sum of sine functions creating a beat frequency; a constant value which is intermittent with time forming a square or rectangular wave; an arithmetic combination, such as the sum, product or ratio, of two or more of the functions or function types; or randomness; and
a conductor configured to apply the output signal to a biological material to promote a therapeutic effect in the biological material.

20. The system of claim 19, in which said pulse oscillator generates the pulsed signal such that a plurality of pulses of the pulsed signal pulses alternate between two polarities, each having equal pulse lengths, each of the pulse lengths lying in the range from 1 microsecond to 1000 milliseconds.

21. A system for generating an electrical signal for use in biomedical applications, comprising:
a controller configured to generate a control signal having at least two states alternating in a pattern as a function of time, the pattern comprising a succession of “on” and “off” pulses which recur in a regularly repeating pattern with time;
a pulse oscillator that produces an oscillating pulse which is enabled during the “on” pulses of the control signal and generates a pulsed signal, but is disabled and consumes negligibly little power during the “off” pulses of the control signal;
a circuit coupled to receive the pulsed signal and configured to control at least one of a signal intensity of the pulsed signal, invert a portion of the pulsed signal, and suppress at least one of a direct current (D.C.) component and a frequency component of the pulsed signal, to produce an output signal; and
a conductor coupled to transfer the output signal to a biological material to promote a therapeutic effect in the biological material, the therapeutic effect comprising at least one of an increase in cell proliferation, cell differentiation, rate of organism growth, secretion of a desired product, or speed with which a tissue structure is developed; treatment of a wound, a bone fracture, osteoporosis, acute pain, swelling, or an inflammatory disorder, a repetitive stress injury, osteoarthritis, and rheumatoid arthritis; accelerated healing of at least one wound; an improvement or restoration of nerve function; or relief of a psychological condition.

22. The system of claim 21, wherein at least one of the controller and the pulse oscillator comprises a complementary metal-oxide-semiconductor (CMOS) circuit.

23. The system of claim 21, further comprising a power source that includes a battery.

24. The system of claim 21 wherein the pulse oscillator has a duty cycle at least approximately matching a duty cycle of the output signal.

25. A method for generating bioelectric stimulation signals comprising:
generating one or more control signals by a controller;
generating one or more pulse sequences by a pulse generator in response to the control signals;
halting an operation of the pulse generator from time-to-time in response to the control signals such that the pulse generator consumes negligibly little power when halted; and
processing the pulse sequences to control at least one of an intensity, a polarity, a control charge balance, or an undesirable frequency component.

26. The method of claim 25, further comprising:
coupling the pulse sequences into a biological material to promote a therapeutic effect in the biological material with the pulse sequences.

27. The method of claim 25, wherein the therapeutic effect comprises at least one of: a treatment of one or more bone fractures, a treatment of osteoporosis, a treatment for acute pain, a treatment of swelling, a treatment of an inflammatory disorder, accelerated healing of at least one wound, an improvement or restoration of nerve function, relief of a psychological condition, increased cell proliferation, increased cell differentiation, an increased rate of organism growth, an increased secretion of a desired product, or increasing a speed in which a tissue structure is developed.

28. The method of claim 25, further comprising supplying power to the signal generating device from a direct current (D.C.) power source.
